(19) United States
(12) Patent Application Publication
(10) Pub. No.: US 2019/0247524 A1
(43) Pub. Date: Aug. 15, 2019

(54) POLYMER-METAL OXIDE COMPLEX, PREPARATION METHOD THEREFOR, AND APPLICATIONS

(71) Applicant: JIANGSU NANOFE BIOMEDICAL TECH. CO., LTD., Jiangsu (CN)

(72) Inventor: Liying HOU, Shanghai (CN)

(73) Assignee: JIANGSU NANOFE BIOMEDICAL TECH. CO., LTD., Jiangsu (CN)

(21) Appl. No.: 16/325,604

(22) PCT Filed: Oct. 18, 2017
(86) PCT No.: PCT/CN2017/106676

§ 371 Date: Apr. 29, 2019

(30) Foreign Application Priority Data
Aug. 19, 2016 (CN) 201610695196.X

(51) Int. Cl. A61K 49/18; C08F 292/00; A61P 7/06; A61K 33/26; A61K 9/00; A61K 47/69; A61K 47/58

(52) U.S. Cl. CPC A61K 49/1854; C08F 292/00; A61P 7/06; A61K 47/58; A61K 9/0019; A61K 9/0053; A61K 47/6929; A61K 33/26

(57) ABSTRACT

A polymer-metal oxide complex, comprising a metal oxide particle located at the core and a polymer modified on the surface of the metal oxide particle, the polymer being provided with functional groups capable of bonding with a metal in the metal oxide, the density of the binding sites of the polymer and the surface of the metal oxide particle being greater than two sites/square nanometer. Also disclosed are a preparation method for the polymer-metal oxide complex, and applications of the polymer-metal oxide complex as a nuclear magnetic resonance contrast agent and as an iron supplement. The polymer-metal oxide complex has a significantly extended in vivo circulation time and effectively overcomes the defect that existing contrast agents cause hypersensitivity, which in addition to the superparamagnetic and iron metabolism participation functions of the complex, enables the complex to be applied as a magnetic resonance imaging contrast agent and as an iron supplement treating iron-deficiency anaemia.

The present application claims priority of Chinese Patent Application No. 201610695196.X, filed on Aug. 19, 2016, the contents of which are incorporated herein by reference in their entireties.

### FIELD OF INVENTION

The present invention relates to a field of nanotechnology and biomedical engineering, in particular to a polymer-metal oxide complex.

### PRIOR ARTS

In recent years, superparamagnetic nanoparticles typically represented by iron oxide have been widely applied in various fields of biomedical science such as biomagnetic separation, targeted drug delivery, gene transfection, immunodiagnosis, iron-deficiency anaemia treatment, enhanced magnetic resonance imaging and the like, owing to their unique physical and chemical properties. In the above fields, especially iron oxide nanoparticles have been widely studied and applied in superparamagnetic magnetic resonance imaging contrast agents and the treatment of iron-deficiency anaemia.

Currently, the molecular structure of iron oxide nanoparticles in the above uses is mainly a polymer complex of ferroferric oxide or ferric oxide. The commercial superparamagnetic iron oxide complex imaging contrast agents mainly comprise Combidex, Resovist and Feridex. The molecular structure of these three types of iron oxide complexes comprises a ferroferric oxide crystal particle located at the core, on the surface of which the hydroxyl of the hydrophilic polymer (such as dextran) is coordinated to the iron atom to form chelation. Therefore, such iron oxide complexes are able to be dispersed in an aqueous solution. The commercial iron oxide nanoparticles as an iron-deficiency anaemia iron supplement mainly comprise Ferumoxtol, of which the molecular structure is that the carboxylate-modified dextran chelate the iron atom. Due to the weak coordination ability and the low coordination density between the hydrophilic polymer which modifies the surface of the above iron oxide and the central iron atom, it is prone to the loss of dispersion stability caused by the detachment of polymers on the surface and arouse the hypersensitivity reaction caused by the release of free iron ions during processing or storage, especially during sterilization with high temperature steam [Juan Gallo, Nicholas J. Long, Eric O. Aboagye. Chemistry. SOC. Rev 42 (2013) 7816]. The prior commercial Freidex® and Resovist® contrast agents are prepared via coprecipitation method to give a final particle size of 6-150 nm, of which the central iron oxide particles have a diameter of about 5 nm and the surface is coated with dextran to reduce surface biotoxicity and increase particle dispersion stability. Such preparation method has the advantages of simple method, simple operation and accessibility of large-scale manufacturing. However, the reaction rate of the coprecipitation method is relatively fast and the nucleation and crystallization processes are difficult to separate, which results in poor mono-disperse of the particles and wide particle size distribution, thereby requiring further sifting to obtain a desired particle size. Moreover, since the reaction medium of the coprecipitation method is an aqueous phase, the reaction temperature which is lower than 100° C. results in the low crystallinity of the central iron oxide crystals, thereby resulting in weaker magnetization and poorer actual contrast effect. The nanoparticles with narrow particle size distribution and high crystallinity can be prepared via the method of high-temperature thermal decomposition due to the high reaction temperature. The nano-particles obtained via the method of traditional high-temperature thermal decomposition are generally oil-soluble, which is not conducive for further biological uses. Due to the poor surface modification of the nanoparticles in aqueous phase using polyol as a stabilizer, it is prone to poor agglomeration stability in the use of vivo angiography and seriously affects the blood circulation, of which the imaging results are not satisfactory.

U.S. Pat. No. 6,599,498B1 discloses a molecular structure of a superparamagnetic iron oxide complex modified by using carboxyl dextran as a coordination polymer. Such iron oxide complex uses carboxyl dextran as a surface-modified polymer so that the coordination capacity of the central iron atom is enhanced and at the same time the exudation of the free iron ions is reduced during use, which alleviates the hypersensitivity reaction in clinical use [V. S. Balakrishnan et al., Eur. J. Clin. investment. 39 (2009) 489.]. However, the complete chelation of iron atoms on the iron oxide surface is still not achieved with such molecular structure. There are still issues of dispersion stability and hypersensitivity reaction caused by the exudation of the free iron ions in clinical uses.

CN103347543A discloses a molecular structure of an iron oxide complex coated with hydrophilic material. The core of such complex is an iron oxide particle with high crystallinity and the surface is coupled with the carboxylmethyl dextran via ligand exchange. However, due to the hydrophobic surface of the nanoparticle unconducive for biological uses, further ligand exchange is required, which means hydrophilic polymer ligands are required for the conversion of the nanoparticles into hydrophilic nanoparticles. Due to the long circulation and involving the ligand exchange in such method, the chelation between the surface ligands and the iron ions is weak, and the free iron ions are easy to fall off and release to cause a hypersensitivity reaction.

CN101002951A discloses a method for preparing the hydrophilic iron oxide complex molecules via polyol method, wherein it is relatively easy to prepare the hydrophilic, mono-dispersed and high-crystallinity iron oxide complex molecules. However, the physiological stability of the complexes is rather poor so that the characteristics of blood circulation in the living body are unable to be guaranteed.

Therefore, the design of a molecular structure of the iron oxide complex, the complete chelation of the iron atoms on the surface of the iron oxide nanoparticles, and the high-density external hydrophilic groups of the complex, have become the key issues in solving the molecular structure design of superparamagnetic iron oxide complex and the in vivo use thereof, which are not only related to the use effect of the iron oxide complex as a magnetic resonance imaging contrast agent and iron-deficiency iron supplement in vivo, but also to the resolution of safety issues during the use in vivo, such as hypersensitivity reactions caused by the release of the free iron ions.

### Content of the Present Invention

In view of the deficiencies of the prior art mentioned above, the present invention provides a polymer-metal oxide complex, wherein the polymer-metal oxide complex comprising a metal oxide particle located at the core and polymers modified on the surface of the metal oxide particle. The polymer is provided with functional groups capable of bonding to the metal in the metal oxide. The binding sites density of the polymers binding to the surface of the metal oxide particle is greater than 2 sites/nm². The general molecular formula of the polymer-metal oxide complex is MₙNₚOₘCₐH_{b}Na_{c}, wherein M represents a metal element; N is N, P or S; n is 500-20000; p is 0-20000; a is 1000-50000; c is 500-20000; m=(3/2−4/3)n+(2/3)a and b=(4/3)a.

Further, the polymer-metal oxide complex comprises one or more than one metal elements such as iron, cobalt, nickel, iron-cobalt, iron-nickel, and the like.

Further, the polymer is selected from the group consisting of polyacrylic acid, polyacrylate salt, methyl polyacrylic acid, methyl polyacrylate, polylactic acid, polylactic acid salt and polyphosphoric ester.

Further, the weight average molecular weight of the polymer is 500-500,000 Da.

Even further, the weight average molecular weight of the polymer is 500-3,000 Da. The polymer-metal oxide complex with a lower molecular weight is less toxic to organisms and provides enhanced biocompatibility when used as injection.

Further, the polymer accounts for 25%-70% of the total weight of the polymer-metal oxide complex.

Further, the polymer accounts for 40%-70% of the total weight of the polymer-metal oxide complex.

Further, the metal oxide is selected from the group consisting of iron oxide, manganese oxide, cobalt oxide, chromium oxide, and nickel oxide.

Further, the metal oxide is iron oxide.

Further, the polymer-metal oxide complex is a polyacrylic acid-iron oxide complex, and the binding sites density of the polyacrylic acid and the surface of the iron oxide particles is greater than 2 sites/nm²; the general molecular formula of the polyacrylic acid-iron oxide complex is: FeₙOₘCₐH_{b}Na_{c}, wherein n is 500-20000; c is 500-20000; m=(3/2-4/3)n+(2/3)a and b=(4/3)a.

Further, the diameter of the central iron oxide particle is 1-30 nm determined by a transmission electron microscope.

Further, the surface-coupled polymer is a polyacrylic acid with a low molecular weight. The weight average molecular weight of the polyacrylic acid is 1000-10000, and the polyacrylic acid accounts for 25%-70% of the total weight of the complex molecule.

The polyacrylic acid-iron oxide complex consists of an iron oxide core with high crystallinity and a high proportion of surface carboxyl polymers. The novel molecular structure provides the polyacrylic acid-iron oxide complex with high degree of hydrophilicity, high dispersion stability in the saline solution, good chelating properties with free and surface iron ions, excellent magnetic resonance relaxation enhancement properties and iron metabolism properties. The above properties enable the novel polyacrylic acid-iron oxide complexes to be used in the magnetic resonance imaging contrast agents for the tissues or cells, such as blood vessels, liver, spleen, lymph, and heart, and in the field of iron supplements for the iron-deficiency anaemia.

The present invention also provides a preparation method of the above polymer-metal oxide complex, which comprises the following steps:

(i) Solution B was prepared by dissolving the precipitation agent in a reducing solvent.

(ii) The polymer was dissolved in the reducing solvent.

(iii) The metal salt was weighed and dissolved in the mixed solution according to (ii) to prepare solution A.

(iv) The reaction of the solution A and the solution B were carried out under microwave condition, followed by cooling to give the polymer-metal oxide complex molecular colloid.

(v) The polymer-metal oxide complex molecular colloid according to (iv) was isolated and rinsed to remove impurities (Removing impurities mainly means removing the solvent, the heavy metals or the unreacted polymers, etc.) to give the polymer-metal oxide complex.

Further, the reducing solvent is a hydrophilic high-boiling-point solvent, and the boiling point thereof is no lower than 180° C.

Further, the reducing solvent is selected from the group consisting of diethylene glycol, ethylene glycol, 1,3-propanediol, glycerin, 1,2-propanediol, and diethylene glycol

Further, the precipitation agent is sodium hydroxide, sodium acetate or sodium borohydride.

Further, the polymer is selected from the group consisting of polyacrylic acid, polyacrylate salt, methyl polyacrylic acid, methyl polyacrylate, polylactic acid, polylactic acid salt, and polyphosphoric ester.

Further, the metal salt is selected from the group consisting of ferric chloride, ferric sulfate, ferric hydroxide, iron (III) 2,4-pentanedionate, and iron (III) cobalt (II) acetylacetonate.

Further, the reaction temperature under the microwave condition is 180° C.-280° C.

Further, the reaction time under the microwave condition is 5 min-30 min.

Further, the preparation method for the polyacrylic acid-iron oxide complex, which comprises the following steps:

(i) Solution B was prepared by dissolving the precipitation agent in a reducing solvent.

(ii) The polyacrylic acid was dissolved in the reducing solvent.

(iii) The iron salt was weighed and dissolved in the mixed solution according to (ii) to prepare solution A.

(iv) The reaction of the solution A and the solution B were carried out under microwave condition, followed by cooling to give the polymer-metal oxide complex colloid.

(v) The polyacrylic acid-iron oxide complex colloid according to (iv) was isolated and rinsed to remove impurities to give the polyacrylic acid-iron oxide complex.

The present invention also provides a nuclear magnetic resonance imaging contrast agent comprising the above polymer-metal oxide complex containing iron element. The nuclear magnetic resonance imaging contrast agent can be used for the enhanced T1, T2 and T2* magnetic resonance imaging can be performed for normal or diseased blood vessels, liver, spleen, lymph, heart and other organs or tissues.

Further, the nuclear magnetic resonance imaging contrast agent is for injection or oral administration.

The invention also provides a use of the above nuclear magnetic resonance imaging contrast agent in the nuclear magnetic imaging of tissues or cells.

Further, the tissues or cells are blood vessels, livers, spleen lymph or hearts.

The present invention also provides an iron supplement comprising the above polymer-metal oxide complex containing the iron element. The iron supplement can rapidly increase the levels of hemoglobin and transferrin in the blood.

Further, the iron supplement is for injection or oral administration. The oral administration comprises capsules, tablets and the like.

The present invention also provides a use of the above iron supplement in the preparation of a medicament for treating iron-deficiency anaemia.

The polymer-metal oxide complex of the present invention contains a high amount of polyelectrolytes with good dispersion stability, uniform particle size distribution and high crystallinity of the central metal oxide, good contrast effect of imaging and long-circulating function. Moreover, the synthesis process is shortened by the microwave-assistant owing to the satisfactory uniformity of microwave heating and high heating efficiency, thereby reducing the preparation cost. The present invention provides a novel molecular structure of a polymer-metal oxide, especially a polyacrylic acid-iron oxide complex, and a preparation method thereof for overcoming the defects of the low chelation density and weak bonding strength of the surface iron atoms in the prior iron oxide complexes. The complex is possessed into various dosage forms for the uses in magnetic resonance imaging contrast agents and iron supplements for treating iron-deficiency anaemia. The polymer-metal oxide complex has significantly prolonged in vivo circulation time and effectively overcome the defect of the hypersensitivity caused by the prior contrast agents. Furthermore, the superparamagnetic property and the function of participating in the iron metabolism of the complex enable the complex to be used as a magnetic resonance imaging contrast agent and an iron supplement treating iron-deficiency anaemia.

The present invention will be further described with reference to the drawings in order to fully explain the objects, technical features and technical effects of the present invention.

### BRIEF DESCRIPTION OF THE DRAWINGS

The objects, technical features and technical effects of the present invention are obvious according to the description of the preferred embodiments with reference to the drawings, wherein:

FIG. 1 are the TGA curves of four polyacrylic acid-iron oxide complexes in the preferred embodiments of the present invention. FIG. 1a to 1d are the TGA curves of the polyacrylic acid-iron oxide complexes according to the preferred embodiments 1 to 4, respectively.

FIG. 2 are the results of the transmission electron microscope (TEM) imaging and the particle size distribution diagrams of four polyacrylic acid-iron oxide complexes in the preferred embodiments of the present invention. FIG. 2a to 2d are the results of the transmission electron microscope (TEM) imaging and the particle size distribution diagrams according to the preferred embodiments 1 to 4, respectively.

FIG. 3 is the saturation magnetization curve of four polyacrylic acid-iron oxide complexes in the preferred embodiments of the present invention. FIG. 3a is the saturation magnetization curve of four polyacrylic acid-iron oxide complexes according to the preferred embodiments 1 to 4. FIG. 3b is an enlarged view of the curve near zero.

FIG. 4 are the relaxation time curves of four polyacrylic acid-iron oxide complexes in the preferred embodiments of the present invention. FIGS. 4a and 4b are the relaxation time curves of four polyacrylic acid-iron oxide complexes according to the preferred embodiments 1 to 4.

FIG. 5 is the sterilization stability curve of four polyacrylic acid-iron oxide complex molecular injections according to the preferred embodiments of the present invention.

FIG. 6 is the stability curve of four polyacrylic acid-iron oxide complex molecular injections according to the preferred embodiments of the present invention.

FIG. 7 are the results of the magnetic resonance imaging of a normal liver with different concentration of polyacrylic acid-iron oxide complex molecular injections according to the preferred embodiments of the present invention.

FIG. 8 is the comparison of the magnetic resonance imaging of a normal blood vessel between the polyacrylic acid-iron oxide complex molecular injections according to the preferred embodiments of the present invention and a commercial sputum contrast agent.

FIG. 9 are the results of the magnetic resonance imaging of a diseased liver tissue (liver cancer) with the polyacrylic acid-iron oxide complex molecular injections according to the preferred embodiments of the present invention.

FIG. 10 are the results of the magnetic resonance imaging of a diseased blood vessel (aneurysm) with the polyacrylic acid-iron oxide complex molecular injections according to the preferred embodiments of the present invention. The hemangioma bridging with the left common carotid artery of the aneurysm model rabbit (shown as the head of the arrow) is clearly displayed in FIG. 10a, wherein the frontal view of the hemangioma in the middle part of the left common carotid artery of the aneurysm model rabbit shows that the diameter of the hemangioma is significantly larger than that of the common carotid artery, which is significantly different from the normal contralateral carotid artery. The aneurysm is displayed more clearly after the rest of blood vessels are removed in FIG. 10b. The lateral views of the aneurysm in FIG. 10c and FIG. 10d are highly consistent with the pathological morphology of the aneurysm taken during surgery.

FIG. 11 are the results of the magnetic resonance imaging of the coronary artery of the polyacrylic acid-iron oxide complex molecular injections according to the preferred embodiments of the present invention. FIG. 11a is the image of the coronary artery after 15 min (shown as a dashed line), wherein the coronary artery can be clearly displayed. FIG. 11b is the image of the coronary artery after 180 min (shown as a dashed line), wherein the coronary artery has almost disappeared. FIG. 11c is the image of the anterior descending coronary artery after 15 min (shown as a dashed line), wherein the anterior descending coronary artery can be clearly displayed. FIG. 1d is the image of the circumflex branch of the coronary artery after 15 min (shown as a dashed line), wherein the circumflex branch of the coronary artery can be clearly displayed.

FIG. 12 is a schematic of the possible structure of the polyacrylic acid-iron oxide complex molecule according to the preferred embodiments of the present invention.

### DETAILED DESCRIPTION OF THE PREFERRED EMBODIMENT

In order to further illustrate the present invention and understand the technical solution thereof, the exemplary but non-limited embodiments are shown as followed:

### Embodiment 1

Synthesis Route:

Firstly, 20 ml of diethylene glycol was measured with a graduated cylinder, and 2 g of sodium hydroxide was weighed with a precision balance, dissolved in diethylene glycol via ultrasonication with heating and stirring to prepare solution B. The solution B was placed in an oven with a constant temperature of 70° C. Secondly, 6 g of polyacrylic acid with a weight average molecular weight of 1000 Da and 500 ml of diethylene glycol were weighed in a beaker, followed by dissolving via ultrasonication with stirring. Sequentially, 2 g of anhydrous iron(III) chloride was weighed and dissolved in the mixed solution of diethylene glycol and polyacrylic acid via ultrasonication with heating and stirring to prepare the solution A (brown color). Finally, the solution A was placed in a three-necked flask and kept in a microwave reactor with a constant temperature of 200° C. for 20 min, and then the hot solution B was rapidly added thereto. The reaction was carried out instantaneously with the constant temperature for 30 min. Thereafter, the reaction mixture was cooled to obtain a polyacrylic acid-iron oxide complex colloid, followed by precipitation with ethyl acetate. The precipitated complex was rinsed three times to give a polyacrylic acid-iron(II,III) oxide complex.

### Embodiment 2

Synthesis Route:

Firstly, 100 ml of ethylene glycol was measured with a graduated cylinder, and 8 g of sodium acetate was weighed with a precision balance, dissolved in ethylene glycol via ultrasonication with heating and stirring to prepare solution B. The solution B was placed in an oven with a constant temperature of 70° C. Secondly, 12 g of polyacrylic acid with a weight average molecular weight of 5000 Da and 360 ml of ethylene glycol were weighed in a beaker, followed by dissolving via ultrasonication with stirring. Sequentially, 8 g of iron(III) sulfate was weighed and dissolved in the mixed solution of ethylene glycol and polyacrylic acid via ultrasonication with heating and stirring to prepare the solution A (brown color). Finally, the solution A was placed in a three-necked flask and kept in a microwave reactor with a constant temperature of 220° C. for 10 min, and then the hot solution B was rapidly added thereto. The reaction was carried out instantaneously with the constant temperature for 10 min. Thereafter, the reaction mixture was cooled to obtain a polyacrylic acid-iron oxide complex colloid. The complex colloid was rinsed with 4 L of ultrapure water, followed by spraying drying to give a polyacrylic acid-iron(II,III) oxide complex.

### Embodiment 3

Synthesis Route:

Firstly, 100 ml of 1,3-propanediol was measured with a graduated cylinder, and 15 g of sodium borohydride was weighed with a precision balance, dissolved in 1,3-propanediol via ultrasonication with heating and stirring to prepare solution B. The solution B was placed in an oven with a constant temperature of 70° C. Secondly, 30 g of polyacrylic acid with a weight average molecular weight of 3000 Da and 1000 ml of 1,3-propanediol were weighed in a beaker, followed by dissolving via ultrasonication with stirring. Sequentially, 30 g of iron(III) hydroxide was weighed and dissolved in the mixed solution of 1,3-propanediol and polyacrylic acid via ultrasonication with heating and stirring to prepare the solution A (brown color). Finally, the solution A was placed in a three-necked flask and kept in a microwave reactor with a constant temperature of 240° C. for 20 min, and then the hot solution B was rapidly added thereto. The reaction was carried out instantaneously with the constant temperature for 30 min. Thereafter, the reaction mixture was cooled to obtain a polyacrylic acid-iron oxide complex colloid, followed by precipitation with ethyl acetate and ethanol. The precipitated complex was rinsed three times and dispersed in water, followed by lyophilisation to give a polyacrylic acid-iron(II,III) oxide complex.

### Embodiment 4

Synthesis Route:

Firstly, 80 ml of diethylene glycol was measured with a graduated cylinder, and 8 g of sodium hydroxide was weighed with a precision balance, dissolved in diethylene glycol via ultrasonication with heating and stirring to prepare solution B. The solution B was placed in an oven with a constant temperature of 72° C. Secondly, 13.8 g of polyacrylic acid with a weight average molecular weight of 10000 Da and 360 ml of diethylene glycol were weighed in a beaker, followed by dissolving via ultrasonication with stirring. Sequentially, 8 g of anhydrous iron(III) chloride was weighed and dissolved in the mixed solution of diethylene glycol and polyacrylic acid via ultrasonication with heating and stirring to prepare the solution A (brown color). Finally, the solution A was placed in a three-necked flask and kept in a microwave reactor with a constant temperature of 220° C. for 5 min, and then the hot solution B was rapidly added thereto. The reaction was carried out instantaneously with the constant temperature for 10 min. Thereafter, the reaction mixture was cooled to give a polyacrylic acid-iron oxide complex colloid, followed by precipitation with ethyl acetate. The precipitated complex was rinsed three times to give a polyacrylic acid-iron(II,III) oxide complex.

### Embodiment 5

Synthesis Route:

Firstly, 20 ml of ethylene glycol was measured with a graduated cylinder, and 2 g of sodium hydroxide was weighed with a precision balance, dissolved in ethylene glycol via ultrasonication with heating and stirring to prepare solution B. The solution B was placed in an oven with a constant temperature of 70° C. Secondly, 4 g of sodium polyacrylate with a weight average molecular weight of 500 Da and 500 ml of ethylene glycol were weighed in a beaker, followed by dissolving via ultrasonication with stirring. Sequentially, 2 g of anhydrous iron(III) chloride was weighed and dissolved in the mixed solution of ethylene glycol and sodium polyacrylate via ultrasonication with heating and stirring to prepare the solution A (brown color). Finally, the solution A was placed in a three-necked flask and kept in a microwave reactor with a constant temperature of 200° C. for 20 min, and then the hot solution B was rapidly added thereto. The reaction was carried out instantaneously with the constant temperature for 30 min. Thereafter, the reaction mixture was cooled to give a black sodium polyacrylate-iron oxide complex colloid, followed by precipitation with ethyl acetate. The precipitated complex was rinsed three times to give a black sodium polyacrylate-iron(II,III) oxide complex solution. The given sodium polyacrylate-iron(II,III) oxide complex solution was heated to 80° C. and the air was introduced to the system. The reaction was carried out for 4 hours to give a reddish brown sodium polyacrylate-iron(III) oxide complex solution so that the complex was more stable.

### Embodiment 6

Synthesis Route:

Firstly, 20 ml of 1,3-propanediol was measured with a graduated cylinder, and 2 g of sodium hydroxide was weighed with a precision balance, dissolved in 1,3-propanediol via ultrasonication with heating and stirring to prepare solution B. The solution B was placed in an oven with a constant temperature of 70° C. Secondly, 5.6 g of sodium polyacrylate with a weight average molecular weight of 500000 Da and 500 ml of 1,3-propanediol were weighed in a beaker, followed by dissolving via ultrasonication with stirring. Sequentially, 2 g of anhydrous iron(III) chloride was weighed and dissolved in the mixed solution of 1,3-propanediol and sodium polyacrylate via ultrasonication with heating and stirring to prepare the solution A (brown color). Finally, the solution A was placed in a three-necked flask and kept in a microwave reactor with a constant temperature of 200° C. for 20 min, and then the hot solution B was rapidly added thereto. The reaction was carried out instantaneously with the constant temperature for 30 min. Thereafter, the reaction mixture was cooled to give a polyacrylic acid-iron oxide complex colloid, followed by precipitation with ethyl acetate. The precipitated complex was rinsed three times to give a black sodium polyacrylate-iron(II,III) oxide complex solution. The given sodium polyacrylate-iron(II,III) oxide complex solution was heated to 80° C. and the air was introduced to the system. The reaction was carried out for 4 hours to give a reddish brown sodium polyacrylate-iron(III) oxide complex solution so that the complex was more stable.

### Embodiment 7

Synthesis Route:

Firstly, 30 ml of glycerin was measured with a graduated cylinder, and 3 g of sodium hydroxide was weighed with a precision balance, dissolved in glycerin via ultrasonication with heating and stirring to prepare solution B. The solution B was placed in an oven with a constant temperature of 50° C. Secondly, 6 g of the sodium polyacrylate solution (45%) with a weight average molecular weight of 1200 Da and 300 ml of glycerin were weighed in a beaker, followed by dissolving via ultrasonication with stirring. Sequentially, 2 g of iron acetylacetonate and 1 g of iron manganese acetylacetonate was weighed and dissolved in the mixed solution of glycerin and sodium polyacrylate via ultrasonication with heating and stirring to prepare the solution A (brown color). Finally, the solution A was placed in a three-necked flask and kept in a microwave reactor with a constant temperature of 220° C. for 20 min, and then the hot solution B was rapidly added thereto. The reaction was carried out instantaneously with the constant temperature for 30 min. Thereafter, the reaction mixture was cooled to give a sodium polyacrylate-iron manganese oxide complex colloid, followed by precipitation with ethyl acetate. The precipitated complex was rinsed three times to give a black sodium polyacrylate-iron manganese oxide complex solution.

### Embodiment 8

Synthesis Route:

Firstly, 30 ml of 1,2-propanediol was measured with a graduated cylinder, and 4 g of sodium hydroxide was weighed with a precision balance, dissolved in 1,2-propanediol via ultrasonication with heating and stirring to prepare solution B. The solution B was placed in an oven with a constant temperature of 50° C. Secondly, 4 g of the sodium methyl polyacrylate solution (40%) with a weight average molecular weight of 200000 Da and 300 ml of 1,2-propanediol were weighed in a beaker, followed by dissolving via ultrasonication with stirring. Sequentially, 1.5 g of iron acetylacetonate and 1 g of iron cobalt acetylacetonate was weighed and dissolved in the mixed solution of 1,2-propanediol and sodium methyl polyacrylate via ultrasonication with heating and stirring to prepare the solution A (brown color). Finally, the solution A was placed in a three-necked flask and kept in a microwave reactor with a constant temperature of 220° C. for 10 min, and then the hot solution B was rapidly added thereto. The reaction was carried out instantaneously with the constant temperature for 30 min. Thereafter, the reaction mixture was cooled to give a sodium methyl polyacrylate-iron cobalt oxide complex colloid, followed by precipitation with ethyl acetate. The precipitated complex was rinsed three times to give a black sodium methyl polyacrylate-iron cobalt oxide complex solution.

### Embodiment 9

Synthesis Route:

Firstly, 30 ml of diethylene glycol was measured with a graduated cylinder, and 4 g of sodium hydroxide was weighed with a precision balance, dissolved in diethylene glycol via ultrasonication with heating and stirring to prepare solution B. The solution B was placed in an oven with a constant temperature of 50° C. Secondly, 5.6 g of polylactic acid with a weight average molecular weight of 10000 Da and 300 ml of diethylene glycol were weighed in a beaker, followed by dissolving via ultrasonication with stirring. Sequentially, 2 g of anhydrous iron(III) chloride was weighed and dissolved in the mixed solution of diethylene glycol and polylactic acid via ultrasonication with heating and stirring to prepare the solution A (brown color). Finally, the solution A was placed in a three-necked flask and kept in a microwave reactor with a constant temperature of 200° C. for 20 min, and then the hot solution B was rapidly added thereto. The reaction was carried out instantaneously with the constant temperature for 30 min. Thereafter, the reaction mixture was cooled to obtain a polylactic acid-iron oxide complex colloid, followed by precipitation with ethyl acetate. The precipitated complex was rinsed three times to give a black polylactic acid-iron(II,III) oxide complex solution. The given polylactic acid-iron(II,III) oxide complex solution was heated to 80° C. and the air was introduced to the system. The reaction was carried out for 4 hours to give a reddish brown polylactic acid-iron(III) oxide complex solution so that the complex was more stable.

### Embodiment 10

Synthesis Route:

Firstly, 30 ml of diethylene glycol was measured with a graduated cylinder, and 4 g of sodium hydroxide was weighed with a precision balance, dissolved in diethylene glycol via ultrasonication with heating and stirring to prepare solution B. The solution B was placed in an oven with a constant temperature of 50° C. Secondly, 4.2 g of polyphosphoric ester with a weight average molecular weight of 30000 Da and 300 ml of diethylene glycol were weighed in a beaker, followed by dissolving via ultrasonication with stirring. Sequentially, 2 g of anhydrous iron(III) chloride was weighed and dissolved in the mixed solution of diethylene glycol and polyphosphoric ester via ultrasonication with heating and stirring to prepare the solution A (brown color). Finally, the solution A was placed in a three-necked flask and kept in a microwave reactor with a constant temperature of 200° C. for 20 min, and then the hot solution B was rapidly added thereto. The reaction was carried out instantaneously with the constant temperature for 30 min. Thereafter, the reaction mixture was cooled to obtain a polyphosphoric ester-iron oxide complex colloid, followed by precipitation with ethyl acetate. The precipitated complex was rinsed three times to give a black polyphosphoric ester-iron(II,III) oxide complex solution. The given polyphosphoric ester-iron(II,III) oxide complex solution was heated to 80° C. and the air was introduced to the system. The reaction was carried out for 4 hours to give a reddish brown polyphosphoric ester-iron(III) oxide complex solution so that the complex was more stable.

### Embodiment 11

The molecular structure of the polymer-metal oxide complex

Analysis of the polyacrylic acid-iron oxide complex according to embodiment 3:

a. The content of polyacrylic acid in the particles is: 58.261/36.172=1.611 (w/w), which means there is 1.611 g of polyacrylic acid in 1 g of Fe₃O₄.

b. The weight of a Fe₃O₄ nano particles with a diameter of 4.5 nm is: 3.14×4.53/6×5.2×10⁻²¹=2.48×10⁻¹⁹ g; and the number of the Fe₃O₄ molecules is: 2.48×10⁻¹⁹/232×6.02×10²³=644.

c. The amount of 4.5 nm particles in 1 g of Fe₃O₄ is: 1/(2.48×10⁻¹⁹)/(6.02×10²³)=6.70×10⁻⁶ mol.

d. The amount of carboxyl groups in the particle-modified polyacrylic acid is: 1.611/72×1000=22.37 mmol, which means there is 22.37 mmol of carboxyl groups in 1 g of Fe₃O₄.

e. The number of carboxyl groups in one particle-modified polyacrylic acid molecule is: 1.611/72/(6.7×10⁻⁶)=3339.60, which means there is 3339.60 carboxyl groups in one particle surface-modified polyacrylic acid molecule.

f. The number of carboxyl groups in one polyacrylic acid chain with a weight average molecular weight of 5000 Da is: 5000/72=69.44.

g. The number of polyacrylic acid chains on the surface of one particle is: 3339.60/69.44=48.

h. The area occupied by one polyacrylic acid chain on the surface of the 4.5 nm particles is: 3.14×4.52/48.09=1.322 nm².

i. The amount of free carboxyl groups in the polyacrylic acid-iron oxide complex molecule determined by conductometric titration is: 18.681 mmol per 1 g of Fe₃O₄, which means 11 sites of carboxyl groups are chelated on 1.322 nm² of particle surface. Therefore, the chelation density of the carboxyl group is 8 sites/nm².

j. The amount of free carboxyl groups in the iron oxide complex determined by conductometric titration is: 18.681 mmol per 1 g of Fe₃O₄, and a particle-modified polyacrylic acid contains 3339.60 carboxyl groups, 2784 of which are free carboxyl groups, and about more than 80% are saturated with sodium. Therefore, the number of Na atoms is 2227 to 2784.

### Embodiment 12

Calculation of the Molecular Formula of the Polymer-Melt Oxide Complex:

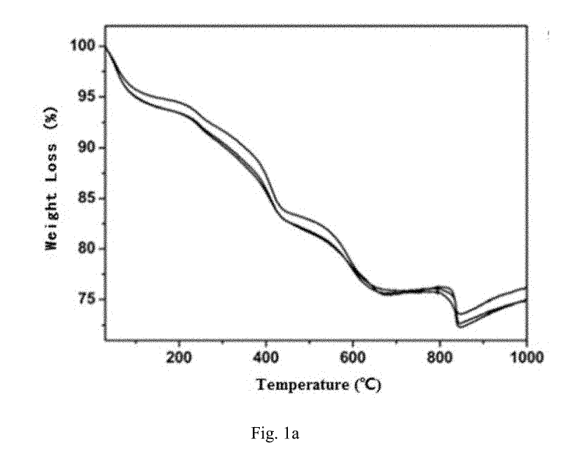

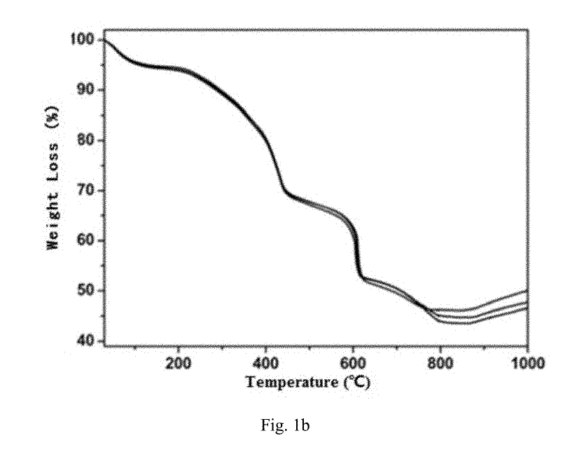

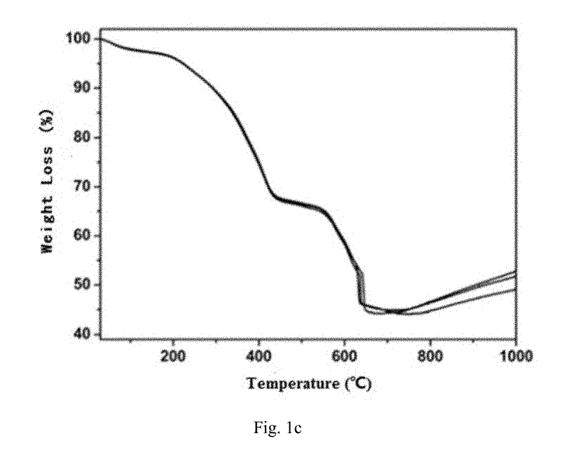

1-2 mL of the polyacrylic acid-iron oxide complex solution according to embodiments 1-4 was lyophilized. 3-5 mg of the lyophilized powder sample was placed in a covered crucible and heated to 1000° C. with a heating rate of 10° C./min. The test instrument used was TG209 from NETZSCH. After the test, the thermogravimetric curves were plotted as shown in FIG. 1, wherein the abscissa was displayed as the temperature, and the ordinate was displayed as the weight loss percentage.

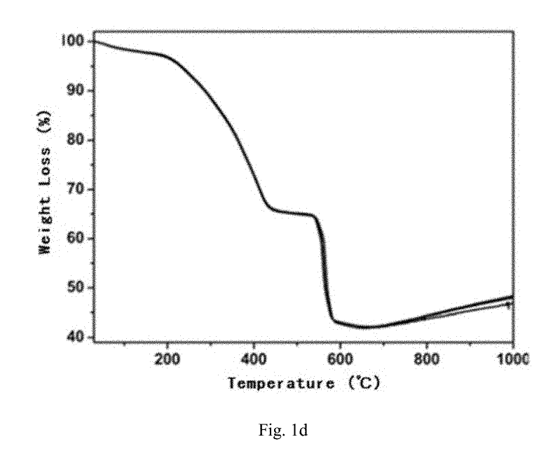

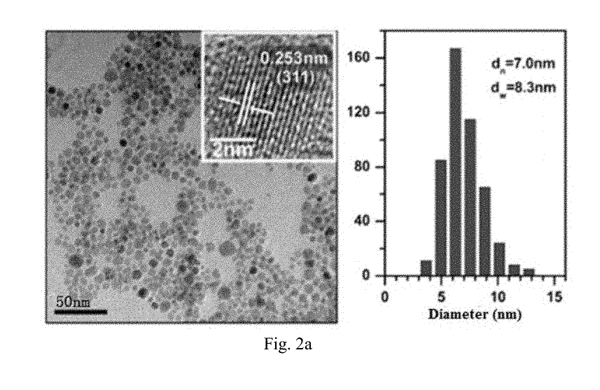

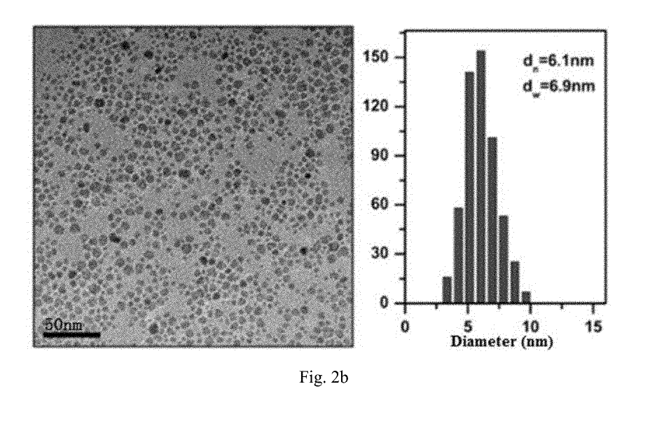

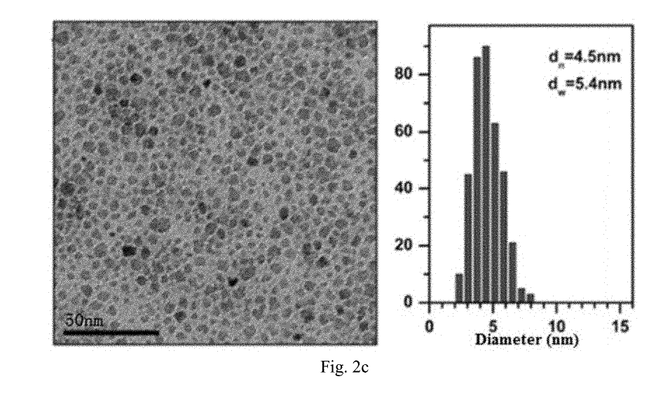

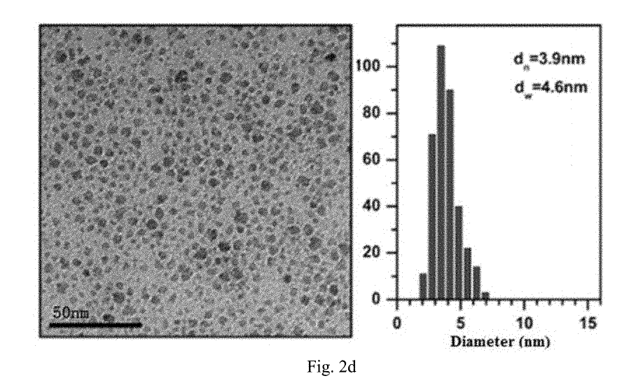

The polyacrylic acid-iron oxide complex solution according to embodiments 1-4 was added on the copper mesh of the carbon support film. The morphology and size of the complex were observed by a transmission electron microscope (TEM) after being naturally dried. The TEM images of the four samples were shown in the left part of FIG. 2. The particle size distribution diagrams of the complexes were shown in the right part of FIG. 2. FIG. 2 shows that the complex is a single crystal nanoparticle molecule, of which the crystallinity is satisfactory. The interplanar spacing is 0.251 nm, corresponding to the crystal plane of iron(II,III) oxide (311). The statistical analysis of the particle size diagrams exhibit that the nano complex molecules have a uniform distribution of the particle size, a high crystallinity and no particle agglomeration.

Calculation of the Molecular Formula of the Complex:

FIG. 2 shows that the diameter of the iron oxide particle according to embodiment 1 is 8.3 nm, and the weight of one complex molecule with a diameter of 8.3 nm is:

$$m = ρ v = ρ \frac{4}{3} {π ( \frac{d}{2} )}^{3} = 5.2 \times {10}^{6} \times \frac{4}{3} \times 3.14 \times {( \frac{8.3 \times {10}^{- 9}}{2} )}^{3} = 1.56 \times {10}^{- 18} g$$

The number of the Fe₃O₄ molecules is: 1.56×10⁻¹⁸×6.02×10²³÷232=4048.

Therefore, the general molecule formula of the polyacrylic acid-iron oxide complex according to embodiment 1 is Fe₁₂₁₄₄O₂₆₀₉₂C₁₄₈₅₀H₁₉₈₀₀.

The general molecule formula of the polyacrylic acid-iron oxide complex according to embodiment 2 is Fe₆₉₆₀O₃₉₆₃₂C₄₅₅₂₈H₆₀₇₀₄, which is determined via the same method.

The general molecule formula of the polyacrylic acid-iron oxide complex according to embodiment 3 is Fe₃₃₃₉O₁₉₀₁₆C₂₁₈₄₆H₂₉₁₂₈.

The general molecule formula of the polyacrylic acid-iron oxide complex according to embodiment 4 is Fe₂₀₆₄O₁₀₇₀₂C₁₁₉₂₅H₁₅₉₀₀.

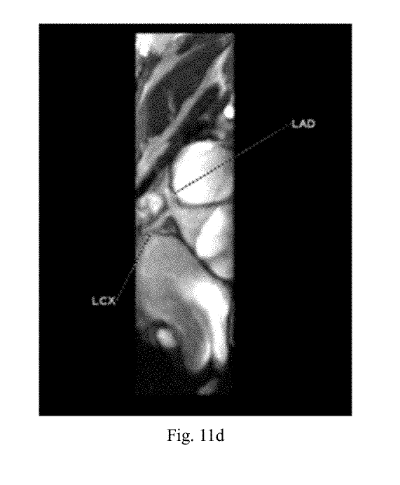

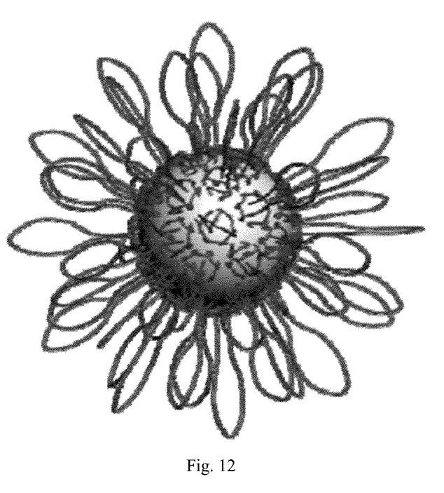

FIG. 12 is the schematic of the possible structure of the polyacrylic acid-iron oxide complex molecule, wherein the core is the grain of iron oxide nanoparticle and the surface is the high-density polyacrylic acid polymer chains. Some carboxyl groups on the molecular chains are bonded to the iron atoms of the iron oxide nanoparticles via chemical coordination bonds. The remaining unchelated carboxyl segments is distributed on the external surface of the particles. The molecular structure of the complex is characterized in that the chelation density of the carboxyl groups with the iron atoms on the surface of the iron oxide is greater than 2 sites/nm². The dense chelation makes the surface of the iron oxide rich in ultra-high amount of polyacrylic acid modifications, which reduce the presence of unchelated surface iron atoms being exposed to the medium, and also greatly improves the bonding stability of the complex, preventing the chelated polyacrylic acid from falling off the surface of the iron oxide. The ultra-high amount of polyacrylic acid modifications also provides the external surface of the complex with a high density of extended carboxyl chain, an excellent hydrophilicity and a high negative potential in aqueous solution, which greatly improve the dispersion stability of the particles. The particle size of the iron oxide nanoparticles of the complex is 1-10 nm determined by the transmission electron microscope, and the weight of the surface-coupled polyacrylic acid accounts for 25%-70% of the total weight of the complex molecules.

### Embodiment 13

Surface Potential and Dispersion Stability of the Polymer-Metal Oxide Complex

Taking the polyacrylic acid-iron oxide complex as an example, there are high amounts of free carboxyl groups on the surface of the polyacrylic acid-iron oxide complex. The surface zeta potential of the polyacrylic acid-iron oxide complex molecules according to embodiments 1-4 in aqueous solution was: −41.3 mV, −42.8 mV, −45.1 mV, and −40.9 mV, respectively, indicating that the complex molecules carry a high amount of negative charges, and these excess negative charges enable the particles to be stably dispersed in the aqueous solution. Subsequently, the polyacrylic acid-iron oxide complexes according to embodiments 1-4 were dispersed in the physiological saline. As shown in the figure, the polyacrylic acid-iron oxide complex molecules were stably dispersed in the physiological saline, wherein the solution was uniform in color and no precipitate was formed, indicating that the negative charges on the surface of the complex molecule enable the molecule to maintain good dispersibility in the physiological saline.

### Embodiment 14

Saturation Magnetization of the Polyacrylic Acid-Iron Oxide Complex Molecules

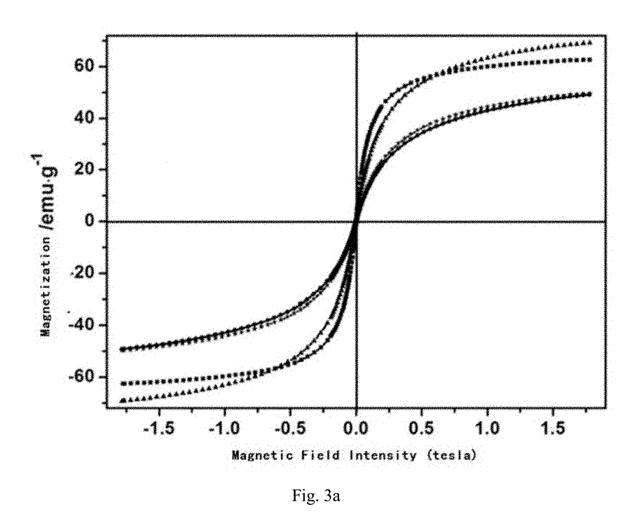

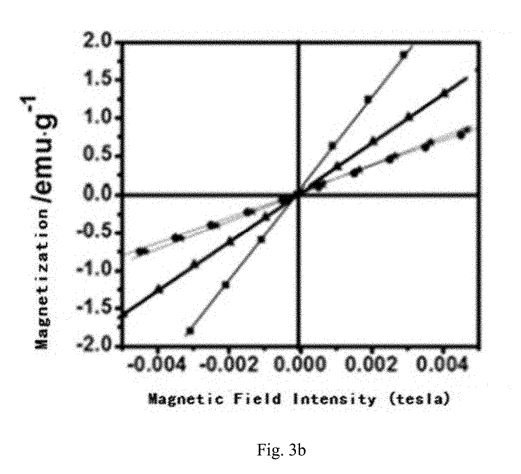

5 mL of the polyacrylic acid-iron oxide complex molecular solution according to embodiments 1-4 was lyophilized and about 10-15 mg of solid powder was weighed before testing. The sample was wrapped into flat rectangular shape with weighing paper. The test was carried out at room temperature using a vibrating sample magnetometer, and the results were shown in FIG. 3, wherein the inserted thumbnails was an enlarged view of the curve near zero. The saturation magnetization of the polyacrylic acid-iron oxide complex molecules according to embodiments 1-4 was: 62.6, 69.4, 49.6, and 49.1 emu/g, respectively. The inserted thumbnails showed that the magnetization curve passed through the origin, which means no remanence and proves that the novel polyacrylic acid-iron oxide complex molecules have superparamagnetism.

### Embodiment 15

Relaxation Properties of Polyacrylic Acid-Iron Oxide Complex Molecules:

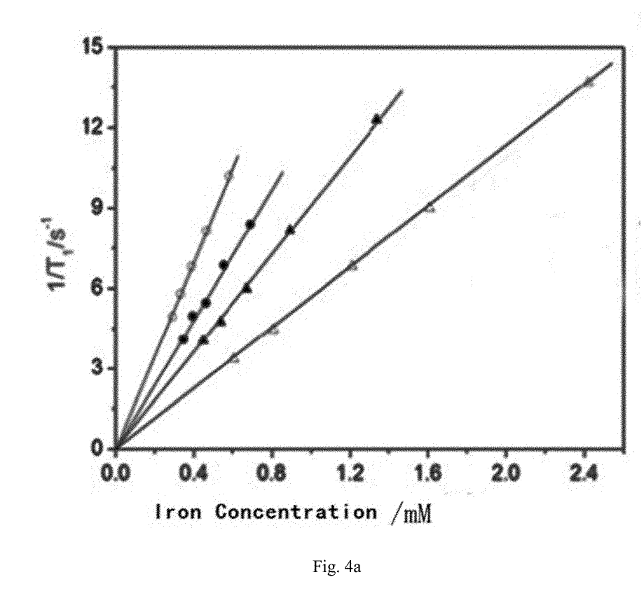

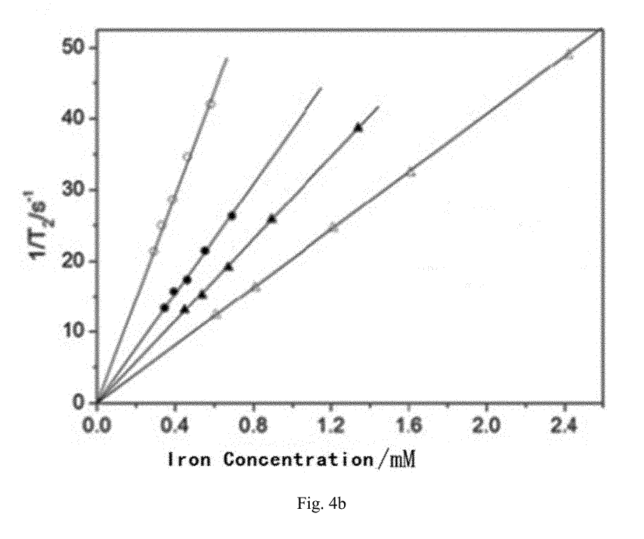

The iron contents of the polyacrylic acid-iron oxide complex molecules according to embodiments 1-4 were measured. Subsequently, the samples were diluted to 4, 5, 6, 7, 8×10⁻⁴ mol/L, and 200 uL of the diluted samples was added into the tubes for the relaxation measurement, numbered 4, 5, 6, 7, and 8, respectively. The tubes were placed in a water bath with a constant temperature of 37° C. Firstly the relaxation time of the samples was measured after calibration. The relaxation time of the polyacrylic acid-iron oxide complex molecules according to embodiments 1-4 was shown in FIG. 4. The relaxation rate r₁ was 5.61-17.5 and the relaxation rate r₂ was 20.3-72.7, wherein the value of r₂/r₁ was 3.2-4.2.

### Embodiment 16

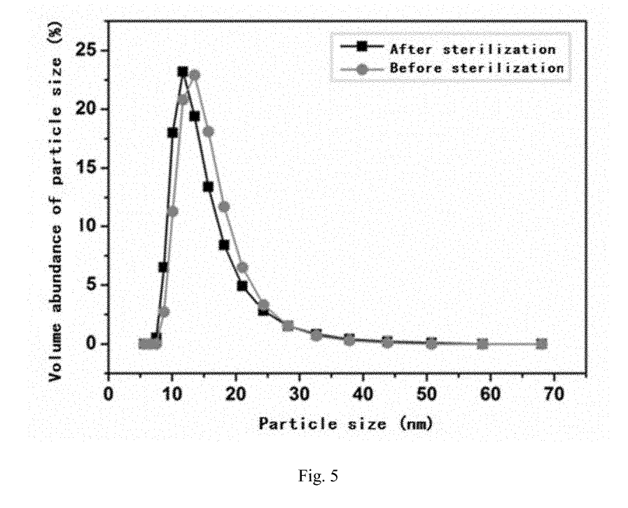

The polyacrylic acid-iron oxide complex molecular solution according to any one of embodiments 1-4 was lyophilized or spray-dried to give a polyacrylic acid-iron oxide complex molecule, and the iron contents were determined by atomic absorption spectrometry. A corresponding volume of water or physiological saline was added according to the final concentration of the iron (umol Fe/L) determined by the above measured iron contents. The final concentration of the iron was 5-1000 umol Fe/L, preferably 50-200 umol Fe/L. The above polyacrylic acid-iron oxide complex molecular solution prepared according to the requirement of the final concentration the iron element is ultrasonically dispersed to give a stable polyacrylic acid-iron oxide complex molecular injection. The given polyacrylic acid-iron oxide complex molecular injection was autoclaved at 121° C. for 30 minutes. The stability of the injection was observed after cooling, wherein no obvious precipitates was formed, and the color of the solution had no significant change. The sample was subjected to dynamic light scattering (DLS) to examine the hydraulic agent of the injection complex molecule after sterilization. As shown in FIG. 5, there was no significant change in particle size and particle size distribution of the polyacrylic acid-iron oxide complex before and after sterilization, indicating that the prepared polyacrylic acid-iron oxide complex molecular injection can be sterilized with high temperature, which effectively improves the safety of the injection.

### Embodiment 17

The polyacrylic acid-iron oxide complex molecular solution according to any one of the embodiments 1-4 was lyophilized or spray-dried to give a polyacrylic acid-iron oxide complex molecule, and the iron contents were determined by atomic absorption spectrometry. Pharmaceutical auxiliary lactose (10%-30%), starch (5%-25%), ethyl cellulose (10%-25%, dissolved in anhydrous ethanol) and anhydrous ethanol were used to prepare the wet granules. The wet granules were passed through 80 mesh stainless steel mesh, dried at room temperature and passed through 20 mesh stainless steel mesh. Then, talcum powder (1%-10%) and stearic acid (0.2%-5%) were added thereto, followed by the even mixing and flat stamping. The final concentration of the iron in the tablet was 5-1000 umol Fe/kg, preferably 50-200 umol Fe/kg.

### Embodiment 18

The polyacrylic acid-iron oxide complex molecular solution according to any one of the embodiments 1-4 was lyophilized or spray-dried to obtain a polyacrylic acid-iron oxide complex molecule, and the iron content was determined by atomic absorption spectrometry. Pharmaceutical excipients lactose (10%-30%), starch (5%-25%), talcum powder (1%-10%) and polysorbate 80 (0.1%-10%) were passed through 80 mesh stainless steel mesh and processed into mixed fine powder. The powder was placed in the empty capsule No. 1. The capsule body was inserted into the powder several times to fill the capsule with a specified weight and sealed using a cap dipped with 40% of ethanol, followed by wipe and polish to obtain the capsule. The final concentration of the iron in the capsule was 5-1000 umol Fe/kg, preferably 50-200 umol Fe/kg.

### Embodiment 19

Stability of the Injections

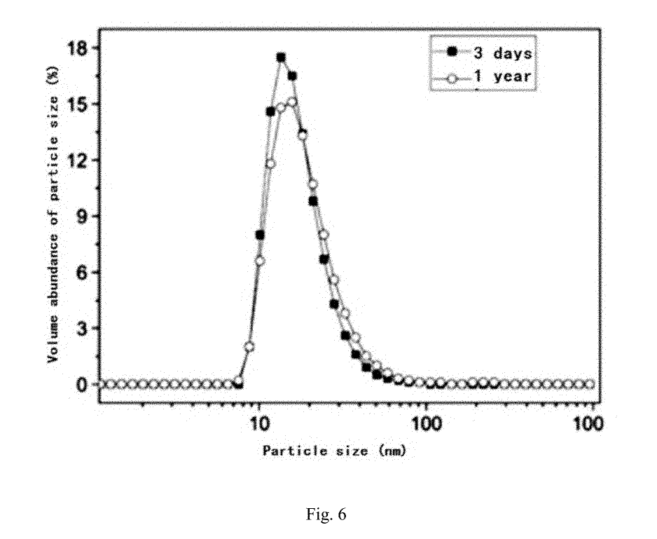

The polyacrylic acid-iron oxide complex molecular injection according to embodiment 16 was placed for three days to one year, and the stability was observed, wherein the color of the injection solution did not change significantly, and no precipitates was formed. The hydraulic diameter of the complex molecules of the injection was determined by dynamic light scattering (DLS). As shown in FIG. 6, there was no significant changes in the hydraulic diameter, indicating that the polyacrylic acid-iron oxide complex molecular has a satisfactory stability and suitable for use as an intravenous injection owing to the high proportion of polyacrylic acid with strong electrostatic repulsion force modified on the surface.

### Embodiment 20

The Release of the Free Iron Ions from the Polyacrylic Acid-Iron Oxide Complex Molecules

Since the free iron ions are the main components causing the hypersensitivity reaction, the polyacrylic acid-iron oxide complex molecular injection according to embodiment 16 was used. The supernatant was filtered with a 3 KDa ultrafiltration centrifuge tube, and the concentration of free iron ions in the supernatant were determined by atomic absorption. A certain amount of free iron ions were also added to the injection and the concentration of free iron ions in the supernatant was determined by atomic absorption after half an hour. The test results were shown in Table 1. The results showed that the free iron ions were not released from the injection even after 120 days. After adding the free iron ions, the injection reduced the concentration of the free iron ions simultaneously, which effectively reduced the hypersensitivity reaction during in vivo imaging. The injection prepared with the novel iron oxide complex molecule can effectively reduce the release of free iron ions and the hypersensitivity reaction, which is suitable for clinical uses as a contrast agent and an iron supplement.

TABLE 1

| | | | Concentration of the | Concentration of the free |
|---|---|---|---|---|
| | Time | | added free iron ions | iron ions in the supernatant |
| 1 | 120 | days | / | 0.24 ug/mL |
| 2 | 2 | hours | / | 0.30 ug/mL |
| 3 | 2 | hours | 300 ug/mL | 8.61 ug/mL |
| 4 | 2 | hours | water | 0.09 ug/mL |

### Embodiment 21

Imaging of a Normal Liver with Different Concentrations of Polyacrylic Acid-Iron Oxide Complex Molecular Injection

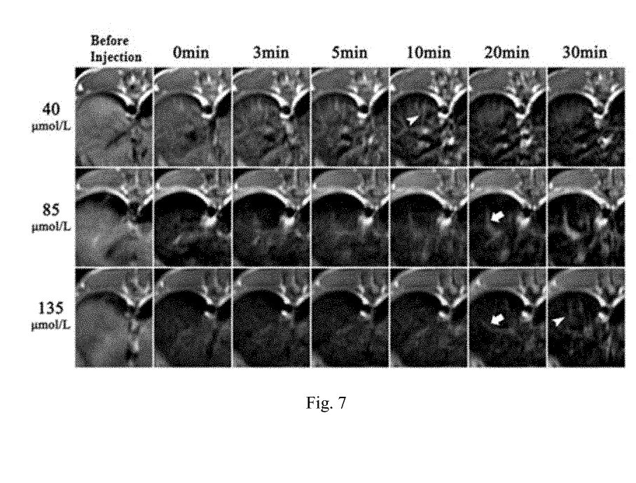

A contrast agent with a concentration of 40, 85, and 135 umol Fe/L was prepared with the polyacrylic acid-iron oxide complex molecular injection according to embodiment 16. The contrast agent was injected into the model rabbit through the ear vein with a dose of 1 ml/kg. T₁ weighted imaging of the model rabbit liver was performed before injection and 0 min, 3 min, 5 min, 10 min, 20 min, 30 min after injection. The results of magnetic resonance imaging were shown in FIG. 7. The signals of model rabbit livers with different injection concentration deceased, wherein the small hepatic vein branches in the model rabbit liver (shown as the head of the arrow) with the 40 umol/L injection was clearly displayed. The main hepatic vein trunk (shown as the head of the arrow) in the model rabbit liver with the 85 umol/L injection was clearly displayed, while the small branches were slightly blurred. The main hepatic vein trunk and small branches in the model rabbit liver with the 135 umol/L injection were displayed, but the imaging results was slightly poorer than the one with 85 umol/L and 40 umol/L, respectively. The results indicates that this novel iron oxide complex molecular injection can be used for magnetic resonance imaging of normal liver tissue.

### Embodiment 22

Magnetic Resonance Imaging of Normal Blood Vessels

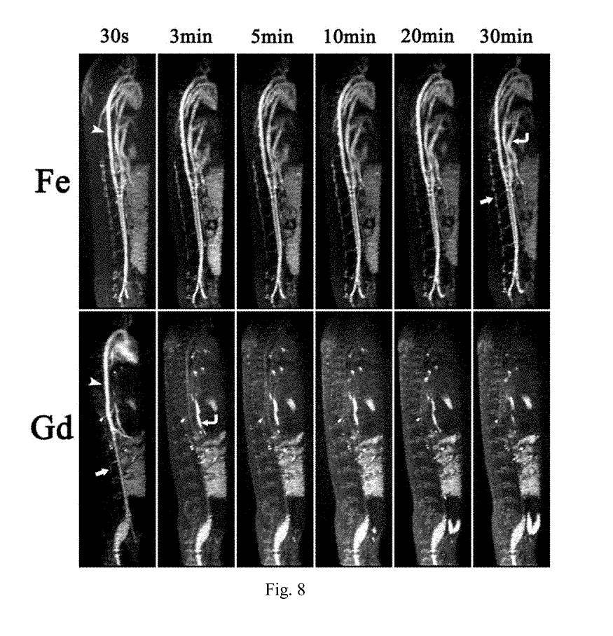

The male white rabbits from New Zealand were used as the test subjects. All rabbits were injected with 2.5% sodium pentobarbital (dose) through the ear vein. The rabbits were fixed on the animal scanning plate in the prone position and pressurized on the abdomen to reduce respiratory artifacts. The contrast agent was injected through the ear vein within 2 s, followed by scanning. The result of magnetic resonance imaging was shown in FIG. 8.

The Agent for Comparison:

A commercial sputum contrast agent was used for comparison: Magnevist® (Gd-DTPA), Bayer Health Care Pharmaceuticals, 469.01 mg/ml×15 ml.

30 seconds after the injection of the stable polyacrylic acid-iron oxide complex molecular injection according to embodiment 16 (concentration: 135 umol/L; dose: 1 ml/kg) and Gd-DTPA, the arteries (indicated by the arrows) were displayed with both iron contrast agent and Gd-DTPA. The portal veins (shown by the curved arrow) were displayed clearly 30 seconds after the injection of the iron contrast agent. However, the best imaging of the portal vein was displayed 3 minutes after the injection of Gd-DTPA and the signal intensity and range were gradually reduced, indicating that the imaging result of the portal vein was significantly worse than the one of the iron contrast agent. The imaging of the aorta and vena cava were not ideal 3 min after the injection of Gd-DTPA, wherein the vascular signal was reduced and the contour was blurred. The aorta, vena cava and portal vein were clearly displayed for at least 30 min after the injection of the iron contrast agent, wherein the imaging results maintained similar throughout the time and the imaging range of the fine spinal cord arteries and veins (shown as thick arrows) slightly increased with the time, while only a small amount of spinal cord arteries were display at 30 s after the injection of Gd-DTPA. It is indicated that that iron contrast agent provides the vascular images with greater contrast. Moreover it is also characterized by the long-circulation so that the blood vessels were still clearly displayed after 30 minutes. The utility model overcomes the defects of the commercial bismuth contrast agent in short development time, unclear signal and the blurred outline. It is indicated that the novel iron oxide complex molecular contrast agent can be applied in long-term vascular imaging in vivo, and the effect of the imaging is far superior to the prior commercial sputum contrast agents owing to its high-density polyacrylic acid coupled on the surface.

### Embodiment 23

Magnetic Resonance Imaging of Liver Cancer Model

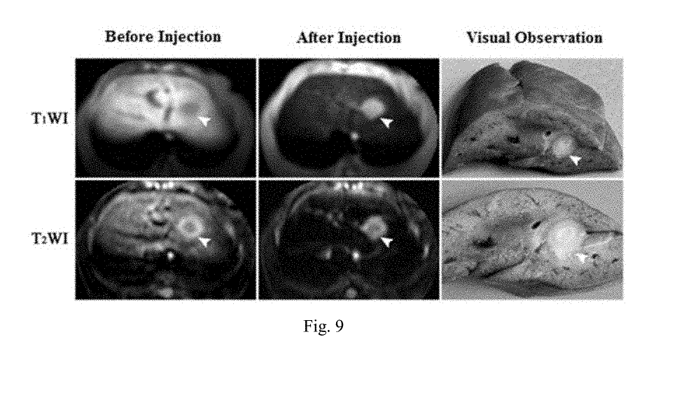

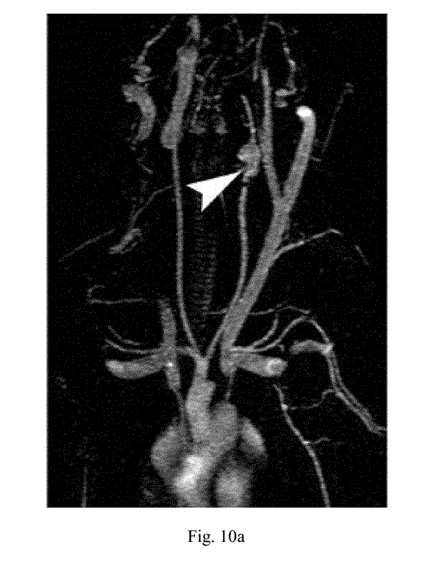

T₁ weighted imaging (T₁WI) and T₂ weighting Imaging (T₂WI) were performed before and after the injection of the stable polyacrylic acid-iron oxide complex according to embodiment 16 to the liver cancer of the model rabbits, respectively. The results of magnetic resonance imaging were shown in FIG. 9. The results of T₁WI and T₂WI imaging on the cancers (shown as the head of the arrow) were more satisfactory than the one before injection, and the liver signals were significantly lower than the one before injection so that the lesions were clearly displayed. Since a low signal of the lesion was generated in the T₁WI before the injection of the contrast agent, the significantly reduced signal of a normal liver generated after the injection of the contrast agent highlighted the imaging of cancer lesion with a relatively high signal, which made the contrast more intense. Compared with the gross pathology of the liver with cancer, the cancer morphology in the enhanced scanning was highly consistent with the cancer lesion morphology. It is indicated that the novel iron oxide complex molecular injection is able to be used for magnetic resonance imaging of diseased liver tissue (such as liver cancer), and the T₁ weighted imaging (T₁WI) and the T₂ weighted imaging (T₂WI) can effectively increase the contrast between the diseased liver tissue and the normal liver tissue, which is conducive to clinical and diagnostic research and has the extremely high potential for clinical uses.

### Embodiment 24

Magnetic Resonance Imaging of Vascular Aneurysm Model

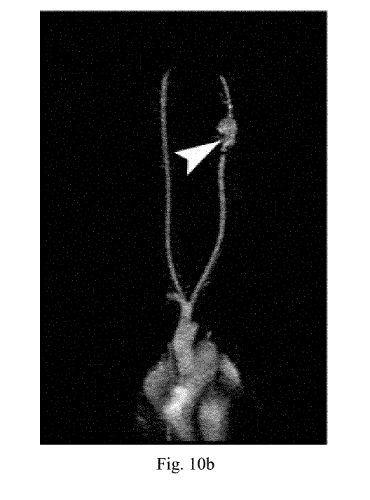

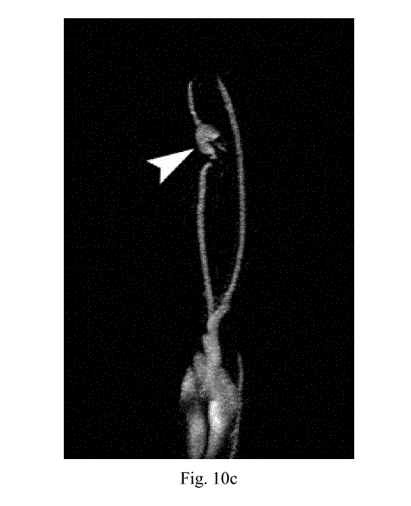

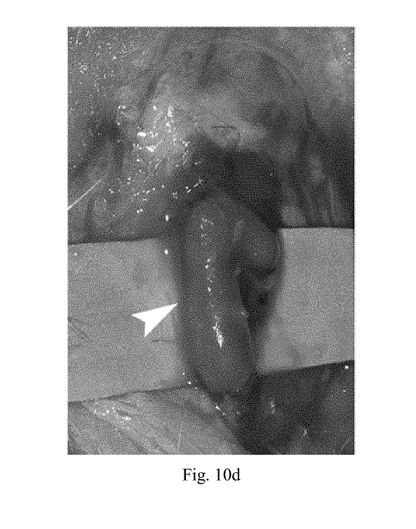

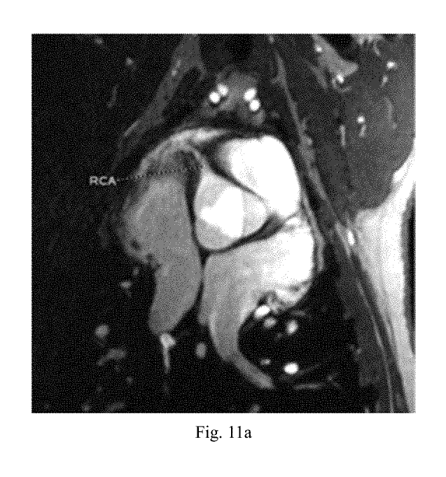

The stabilized polyacrylic acid-iron oxide complex molecular injection according to the embodiment 16 was injected to the liver of the vascular aneurysm model rabbit (concentration: 135 umol/L; dose: 1 ml/kg). The results of magnetic resonance imaging before and after the injections were shown in FIG. 10: a: The hemangioma bridging with the left common carotid artery of the aneurysm model rabbit (shown as the head of the arrow) was clearly displayed, wherein the frontal view of the hemangioma in the middle part of the left common carotid artery of the aneurysm model rabbit showed that the diameter of the hemangioma was significantly larger than that of the common carotid artery, which was significantly different from the normal contralateral carotid artery. b: The aneurysm was displayed more clearly after the rest of blood vessels were removed. c and d: The lateral views of the aneurysm were highly consistent with the pathological morphology of the aneurysm taken during surgery. It is indicated that this new type of iron oxide complex molecular injection can be used for the magnetic resonance imaging of diseased blood vessels (such as aneurysms). Magnetic resonance imaging can effectively increase the contrast between diseased blood vessels and normal blood vessels, which is conducive to the clinical diagnosis and has extremely high potential for the clinical application.

### Embodiment 25

Magnetic Resonance Imaging of the Small Pig Coronary Arteries

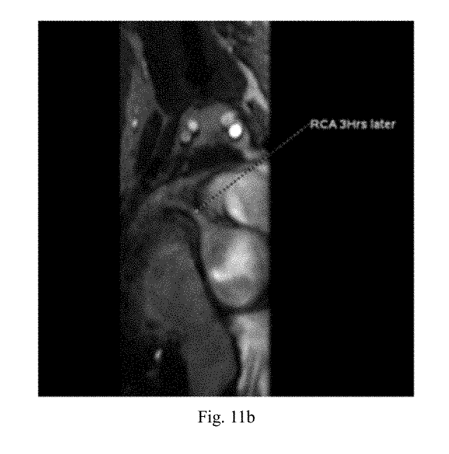

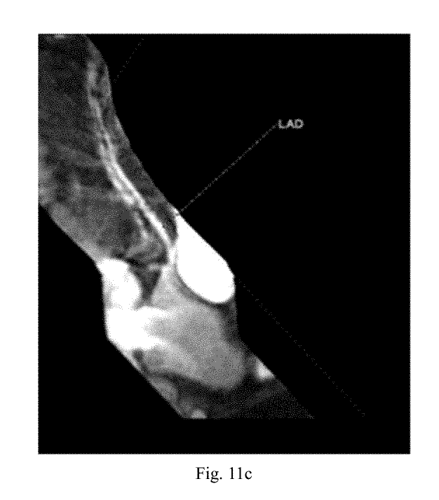

After the small pigs (about 30 kg) were anesthetize by using sodium pentobarbital, the polyacrylic acid-iron oxide complex molecular injection according to embodiment 16 (concentration: 135 umol/L; dose: 1 ml/kg) was intravenously administered to the pigs, followed by imaging using 3T GE magnetic resonance imager (Signa HDxt, 3T). The results of magnetic resonance imaging were shown in FIG. 11, wherein FIG. 11a was the image of the coronary artery after 15 min (shown as a dashed line), wherein the coronary artery was clearly displayed. FIG. 11b was the image of coronary artery after 180 min (shown as a dashed line), wherein the coronary artery had almost disappeared. FIG. 11c was the image of the anterior descending coronary artery after 15 min (shown as a dashed line), wherein the anterior descending coronary artery was clearly displayed. FIG. 11d was the image of the circumflex branch of the coronary artery after 15 min (shown as a dashed line), wherein the circumflex branch of the coronary artery was clearly displayed. It is indicated that the novel iron oxide complex molecular injections can be used for the magnetic resonance imaging of coronary arteries, which is beneficial to clinical diagnosis and has great potential for clinical application.

### Embodiment 26

Iron Supplement Experiments of the Polyacrylic Acid-Iron Oxide Complex Molecular Injections

Eighteen SD male rats (about 200 g) were used and divided into three groups of 6 rats each, comprising the first group (control group) with normal feeding, the second group (iron-deficiency anaemia control group) with low-iron feeding and the third group (iron-deficiency anaemia treatment experimental group) with low-iron feeding. Blood samples were taken at week 4 and the polyacrylic acid-iron oxide complex molecular injection according to embodiment 16 was injected afterwards. The food intake of each group was approximately the same during the experiment. Observation indicators: Blood samples were taken from the tail tip of the rats for measurement at the beginning of the experiments, Week Two, Week Four and Week Five, respectively. The comparison of Hgb (hemoglobin), Hct (hematocrit), and RBC (red blood cells) of each group was shown in Table 2. The experimental results exhibited that the iron-deficiency anemia model was successfully established 4 weeks after the rats were fed with the iron-deficient diet (P<0.01 compared with the blank group). The iron supplement was injected intravenously and an alleviation of the anemia symptoms was observed one week after administration (P<0.05 compared with the blank group), indicating the iron supplementation effect of the preparation.

TABLE 2

| Time | | |
|---|---|---|
| Week Two | Week Four | Week Five |

Hemoglobin (Hgb) test result (x̅ ± SD)

| | | | |
|---|---|---|---|
| Control Group | 146.83 ± 7.88 | 159.33 ± 5.96 | 152.33 ± 11.02 |
| Iron-deficiency | 142.29 ± 14.26 | 119.71 ± 22.04** | 113.67 ± 10.26* |
| Group | | | |
| Treatment Group | 143.00 ± 9.87 | 102.25 ± 27.42** | 137.67 ± 17.90 |

Hematocrit (Hct) test result (x̅ ± SD)

| | | | |
|---|---|---|---|
| Control Group | 42.72 ± 3.24 | 48.82 ± 2.03 | 45.73 ± 3.09 |
| Iron-deficiency | 42.27 ± 3.98 | 37.13 ± 7.01** | 35.89 ± 2.64* |
| Group | | | |
| Treatment Group | 39.94 ± 3.29 | 31.88 ± 8.97** | 49.30 ± 14.77 |

Red blood cell (RBC) test result (x̅ ± SD)

| | | | |
|---|---|---|---|
| Control Group | 7.42 ± 0.65 | 8.60 ± 0.51 | 8.06 ± 0.42 |
| Iron-deficiency | 7.06 ± 0.64 | 6.85 ± 1.25** | 7.11 ± 0.30* |
| Group | | | |
| Treatment Group | 6.66 ± 0.59 | 5.92 ± 1.76** | 7.57 ± 1.05 |

Note:
*P < 0.05;
**P < 0.01

It is to be understood that the foregoing description of two preferred embodiments is intended to be purely illustrative of the principles of the invention, rather than exhaustive thereof, and that changes and variations will be apparent to those skilled in the art, and that the present invention is not intended to be limited other than expressly set forth in the following claims.

What is claimed is:

1. A polymer-metal oxide complex, wherein the polymer-metal oxide complex comprising a metal oxide particle located at the core and polymers modified on the surface of the metal oxide particle, the polymer is provided with functional groups capable of bonding to the metal in the metal oxide; the binding sites density of the polymer binding to the surface of the metal oxide particle is greater than 2 sites/nm²; the general molecular formula of the polymer-metal oxide complex is MₙNₚOₘCₐH_{b}Na_{c}, wherein M represents a metal element; N is N, P or S; n is 500-20000; p is 0-20000; a is 1000-50000; c is 500-20000; m=(3/2−4/3)n+(2/3)a and b=(4/3)a.

2. The polymer-metal oxide complex according to claim 1, wherein the polymer-metal oxide complex comprises one or more than one metal elements.

3. The polymer-metal oxide complex according to claim 1, wherein the polymer is selected from the group consisting of polyacrylic acid, polyacrylate salt, methyl polyacrylic acid, methyl polyacrylate, polylactic acid, polylactic acid salt, and polyphosphoric ester.

4. The polymer-metal oxide complex according to claim 1, wherein the weight average molecular weight of the polymer is 500-500,000 Da.

5. The polymer-metal oxide complex according to claim 1, wherein the weight average molecular weight of the polymer is 500-3,000 Da.

6. The polymer-metal oxide complex according to claim 1, wherein the polymer accounts for 25%-70% of the total weight of the polymer-metal oxide complex.

7. The polymer-metal oxide complex according to claim 1, wherein the polymer accounts for 40%-70% of the total weight of the polymer-metal oxide complex.

8. The polymer-metal oxide complex according to claim 1, wherein the metal oxide is selected from the group consisting of iron oxide, manganese oxide, cobalt oxide, chromium oxide, and nickel oxide.

9. (canceled)

10. The polymer-metal oxide complex according to claim 1, wherein the polymer-metal oxide complex is polyacrylic acid-iron oxide complex, and the binding sites density of the polyacrylic acid binding to the surface of the iron oxide particles is greater than 2 sites/nm²; the general molecular formula of the polyacrylic acid-iron oxide complex is: FeₙOₘCₐH_{b}Na_{c}, wherein n is 500-20000; a is 1000-50000, c is 500-20000; m=(3/2−4/3)n+(2/3)a and b=(4/3)a.

11. A preparation method of the polymer-metal oxide complex according to claim 1, which comprises the following steps:
(i) solution B was prepared by dissolving the precipitation agent in a reducing solvent;
(ii) the polymer was dissolved in the reducing solvent;
(iii) the metal salt was weighed and dissolved in the mixed solution according to (ii) to prepare solution A;
(iv) the reaction of the solution A and the solution B were carried out under microwave condition, followed by cooling to give the polymer-metal oxide complex molecular colloid;
(v) the polymer-metal oxide complex molecular colloid according to (iv) was isolated and rinsed to remove impurities to obtain the polymer-metal oxide complex.

12. The preparation method of the polymer-metal oxide complex according to claim 11, wherein the reducing solvent is a hydrophilic high-boiling-point solvent, and the boiling point thereof is no lower than 180° C.

13. The preparation method of the polymer-metal oxide complex according to claim 11, wherein the reducing solvent is selected from the group consisting of diethylene glycol, ethylene glycol, 1,3-propanediol, glycerin, 1,2-propanediol, and diethylene glycol;
and/or, the precipitation agent is sodium hydroxide, sodium acetate or sodium borohydride;
and/or, the polymer is selected from the group consisting of polyacrylic acid, polyacrylate salt, methyl polyacrylic acid, methyl polyacrylate, polylactic acid, polylactic acid salt, and polyphosphoric ester;
and/or, the metal salt is selected from the group consisting of ferric chloride, ferric sulfate, ferric hydroxide, iron (III) 2,4-pentanedionate, and iron (III) cobalt (II) acetylacetonate.

14. (canceled)

15. (canceled)

16. (canceled)

17. The preparation method of the polymer-metal oxide complex according to claim 11, wherein the reaction temperature under the microwave condition is 180° C.-280° C.

18. The preparation method of the polymer-metal oxide complex according to claim 11, wherein the reaction time under the microwave condition is 5 min-30 min.

19. The preparation method of the polymer-metal oxide complex according to claim 11, wherein the preparation method comprises the following steps
(i) solution B was prepared by dissolving the precipitation agent in a reducing solvent;
(ii) the polyacrylic acid was dissolved in the reducing solvent;
(iii) the iron salt was weighed and dissolved in the mixed solution according to (ii) to prepare solution A;
(iv) the reaction of the solution A and the solution B were carried out under microwave condition, followed by cooling to give the polymer-metal oxide complex molecular colloid;
(v) the polyacrylic acid-iron oxide complex molecular colloid according to (iv) was isolated and rinsed to remove impurities to obtain the polyacrylic acid-iron oxide complex.

20. A nuclear magnetic resonance imaging contrast agent, wherein the nuclear magnetic resonance imaging contrast agent comprising the polymer-metal oxide complex according to claim 8.

21. The nuclear magnetic resonance imaging contrast agent according to claim 20, wherein the nuclear magnetic resonance imaging contrast agent is for injection or oral administration.

22. A use of the nuclear magnetic resonance imaging contrast agent according to claim 20 in the nuclear magnetic imaging of tissues or cells.

23. The use according to claim 22, the tissues or cells are blood vessels, livers, spleen lymph or hearts.

24. An iron supplement, wherein the iron supplement comprises the polymer-metal oxide complex according to claim 8.

25. The iron supplement according to claim 24, wherein the iron supplement is for injection or oral administration.

26. A method for treating iron-deficiency anaemia in a subject in need thereof, comprising administering the iron supplement according to claim 24 to the subject.
